(19) United States
(12) Patent Application Publication
(10) Pub. No.: US 2006/0088617 A1
(43) Pub. Date: Apr. 27, 2006

(54) Chocolate composition and method for benefiting the cardiovascular system

(76) Inventor: James Madison McCurry, Rockwall, TX (US)

Correspondence Address: HENRY L. SMITH, JR., 9273 CORNELL CIRCLE, HIGHLANDS RANCH, CO, 80130-4141, US

(21) Appl. No.: 10/971,919

(22) Filed: Oct. 23, 2004

(51) Int. Cl. A61K 36/18

(52) U.S. Cl. 424/776

(57) ABSTRACT

A medicinal or food supplement composition comprised essentially of dark chocolate less most of the fat and sugar and with high levels of chocolate flavonoids, and a method by which the composition is consumed by a person or other mammal in specified doses per day over a period of time, resulting in several benefits to the cardiovascular system.

### STATEMENT REGARDING FEDERALLY SPONSORED RESEARCH AND DEVELOPMENT

None

### BACKGROUND—FIELD OF INVENTION

The field of the invention is compositions and methods for treating the cardiovascular system and improving cardiovascular functioning in mammals, through the use of natural plant-derived materials.

### BACKGROUND—DESCRIPTION OF PRIOR ART

A recent study at the University of California at San Francisco concluded that flavonoid-rich dark chocolate boosts blood vessel function. Small daily doses of flavonoid-rich regular dark chocolate consumed over a two week period improved the blood vessels ability to dilate or expand. The study indicated that a particular flavonoid thought to be beneficial to blood vessel function, epicatechin, was absorbed at high levels in the blood. Actual blood flow was measured in the brachial artery. Journal of the American College of Nutrition, Vol. 23, #3, p. 197-204.

A study has concluded that flavonoids from tea may protect against atherosclerosis. Geleijnse J M, Launer L J, Hofman A, Huibert A P P, Witteman J C M. The Rotterdam Study. Archives of Internal Medicine 1999; 159: 2170-2174.

LDL cholesterol oxidation which causes problems for the cardiovascular system is reduced by cocoa. Lancet 348:1514. Cocoa inhibits blood platelet activation and clotting. American Journal of Clinical Nutrition 72:30-35. Beneficial antioxidants have been found in chocolate. Lancet 348:834.

### SUMMARY OF INVENTION

The invention comprises a composition derived from chocolate for use in improving aspects of, and prevention of problems with, the mammalian cardiovascular system. The composition is essentially dark chocolate (chocolate without milk solids or other diluents) without the normally associated fat, or cocoa butter, and high levels of sugar. The composition is taken by a human or other mammal in multiple doses daily. Beneficial effects from the composition and method are thought to include increased blood flow through blood vessels, reduced blood levels of the harmful variety of cholesterol, inhibition of blood clot formation, promotion of the opening of clogged arteries, reduction of blockage of arteries by cholesterol and other deposits, prevention of oxidation processes in the blood and body which promote deposits of cholesterol and cancer, promotion of flexibility of the veins and arteries, improvement of the ability of arteries and veins to expand (dilate), and prevention of hardening of the arteries. The novelty of the invention includes not only the substantially lowered amounts of fat and sugar, but also the method and dosage of administration.

### OBJECTS AND ADVANTAGES

The objects and advantages of the present invention are:

1. To provide the benefits for the cardiovascular system which have been well documented for dark chocolate which contains a substantially higher flavonoid content than other products.

2. To provide the benefits for the cardiovascular system of consumption of dark chocolate without the fat or cocoa butter which may raise the person's cholesterol or blood fat levels, and without providing the extra empty calories from sugar, both of which are found in ordinary dark chocolate candy.

3. To increase blood vessel function and an improved ability of blood vessels to dilate and to promote beneficial effects in the cardiovascular system in conjunction with a healthy balanced diet and exercise.

4. To decrease the body's inflammatory immune responses which contribute to atherosclerosis.

5. To reduce the accumulation of blood platelets which contribute to the risk of blood clots that produce heart attacks and strokes.

6. To decrease oxidation of low density lipoprotein (LDL) cholesterol, a detrimental process that allows cholesterol to accumulate in blood vessels.

### DESCRIPTION—PREFERRED EMBODIMENT

The dark cocoa powder (containing no milk solids or other diluents) will typically contain, by weight, less than about 1% of fat, less than about 60% of carbohydrates such as sugars, and not less than about 6 to 8% of chocolate flavonoids. (It may be possible to reduce the fat and carbohydrate content even more, and to increase the flavonoid content even more.) The remaining constituents of the cocoa will include about 20 percent protein, and about 12 percent water and ash. The defatted cocoa powder can be obtained from Progressive Laboratories, Inc., 1701 W. Walnut Hill Lane, Irving, TX 75038-7962. The composition would preferably be contained in standard gelatin capsules of a size for convenient swallowing by the person or other mammal. The composition could be contained in hard tablets or other similar pills. It might also be taken in liquid form. Typically 500 mg or 600 mg capsules may be used. The total amount of the composition administered to the human or other mammal per day might range from 1,000 milligrams to 4,000 milligrams, and may be administered in several portions during the day as convenient, coinciding with meals or between meals. The number of days over which the composition is administered could vary from a few days to several weeks or more.

### OPERATION OF THE INVENTION

Chocolate contains a wide variety of chemical substances including substantial amounts of flavonoids. The Inventor believes that the beneficial effect of his composition is due to the use of chocolate with high levels of flavonoids and substantially reduced levels of fat and sugar. The methods of lowering the fat and carbohydrate content are well known to those skilled in the art.

### TESTS

Various researchers, including those mentioned above, have well documented the benefits of flavonoid-rich dark chocolate on the cardiovascular system. Typically those tests used whole dark chocolate which contained typical amounts of fat primarily in the form of cocoa butter, and enough sugar or other sweeteners to offset the natural somewhat harsh taste of unsweetened chocolate. For various health reasons, it seems desirable to administer flavonoid-rich chocolate without much fat and sugar. It is expected that the composition of the present invention will produce beneficial effects similar to those produced by using dark chocolate containing fat and sugar. Persons skilled in the medical arts would have no reason to think that the well-documented beneficial effects of chocolate stem from the fat or sugar, so it is expected that the composition of the present Invention would produce similar beneficial effects as compared to dark chocolate containing standard amounts of fat and sugar; the only question is how much benefit is produced for various quantities of the composition of the Invention. The Inventor is currently conducting tests on humans to prove that his composition and method have beneficial effects for humans comparable to those observed benefits from tests of whole dark chocolate conducted by medical researchers. It is planned that there will be four groups in the test. One group will receive placebos. Three other groups will receive about 1200 milligrams, 2400 milligrams, and 3600 milligrams of the composition per day. The various groups will not know who will be receiving the placebos or the Inventor's composition.

### ADDITIONAL EMBODIMENTS

By various standard techniques, it would be possible to reduce the carbohydrate, protein, and ash and water content of the composition, thereby increasing the flavonoid percentage above about 6 to 8 percent. In addition, it would be possible to add to the cocoa concentrated chocolate flavonoids, thereby also increasing the flavonoid percentage above about 6 to 8 percent. A higher concentration of flavonoids in the composition may increase its cardiovascular benefits, or alternatively may allow the dosage of the composition to be reduced. Therefore, the Inventor may produce additional embodiments of the Invention with flavonoid percentages greater than about 6 to 8 percent if cost effective or desirable.

### CONCLUSIONS, RAMIFICATIONS AND SCOPE

A number of changes are possible to the compositions and methods described above while still remaining within the scope and spirit of the invention. The specifics about the form of the invention described in this application are not intended to be limiting in scope. The scope of the invention is to be determined by the claims and their legal equivalents, not the examples given above.

I claim:

1. A medicinal or food supplement composition to be administered to a human or other mammal to produce beneficial effects on the cardiovascular system of the human or other mammal, including increased blood flow through blood vessels, comprising dark cocoa as powder, liquid, or as contained in pills or capsules, wherein the cocoa has substantially reduced fat compared with whole dark chocolate candy.

2. The composition of claim 1 wherein the content of carbohydrates including sugars is substantially reduced compared to whole dark chocolate.

3. The composition of claim 1 wherein the fat content is less than about 10 percent.

4. The composition of claim 3 wherein the content of carbohydrates is less than about 70 percent.

5. The composition of claim 4 wherein the percentage of fat is less than about 2 percent, and wherein percentage of carbohydrates is less than about 60 percent.

6. The composition of claim 1 wherein the chocolate flavonoid percentage is between about 6 percent and about 8 percent.

7. The composition of claim 2 wherein the chocolate flavonoid percentage is between about 6 percent and about 8 percent.

8. The composition of claim 3 wherein the chocolate flavonoid percentage is between about 6 percent and about 8 percent.

9. The composition of claim 4 wherein the chocolate flavonoid percentage is between about 6 percent and about 8 percent.

10. The composition of claim 5 wherein the chocolate flavonoid percentage is between about 6 percent and about 8 percent.

11. A method of benefiting the cardiovascular system of a human or other mammal comprising administering to the human or mammal by mouth a total amount per day of the composition of claim 1 of between about 1000 milligrams and about 4000 milligrams.

12. A method of benefiting the cardiovascular system of a human or other mammal comprising administering to the human or mammal by mouth a total amount per day of the composition of claim 2 of between about 1000 milligrams and about 4000 milligrams.

13. A method of benefiting the cardiovascular system of a human or other mammal comprising administering to the human or mammal by mouth a total amount per day of the composition of claim 3 of between about 1000 milligrams and about 4000 milligrams.

14. A method of benefiting the cardiovascular system of a human or other mammal comprising administering to the human or mammal by mouth a total amount per day of the composition of claim 4 of between about 1000 milligrams and about 4000 milligrams.

15. A method of benefiting the cardiovascular system of a human or other mammal comprising administering to the human or mammal by mouth a total amount per day of the composition of claim 5 of between about 1000 milligrams and about 4000 milligrams.

16. A method of benefiting the cardiovascular system of a human or other mammal comprising administering to the human or mammal by mouth a total amount per day of the composition of claim 6 of between about 1000 milligrams and about 4000 milligrams.

17. A method of benefiting the cardiovascular system of a human or other mammal comprising administering to the human or mammal by mouth a total amount per day of the composition of claim 7 of between about 1000 milligrams and about 4000 milligrams.

18. A method of benefiting the cardiovascular system of a human or other mammal comprising administering to the human or mammal by mouth a total amount per day of the composition of claim 8 of between about 1000 milligrams and about 4000 milligrams.

19. A method of benefiting the cardiovascular system of a human or other mammal comprising administering to the human or mammal by mouth a total amount per day of the composition of claim 9 of between about 1000 milligrams and about 4000 milligrams.

20. A method of benefiting the cardiovascular system of a human or other mammal comprising administering to the human or mammal by mouth a total amount per day of the composition of claim 10 of between about 1000 milligrams and about 4000 milligrams.

21. The method of claim 11, wherein the daily amount administered is divided into at least 2 separate doses per day.

22. The method of claim 12, wherein the daily amount administered is divided into at least 2 separate doses per day.

23. The method of claim 13, wherein the daily amount administered is divided into at least 2 separate doses per day.

24. The method of claim 14, wherein the daily amount administered is divided into at least 2 separate doses per day.

25. The method of claim 15, wherein the daily amount administered is divided into at least 2 separate doses per day.

26. The method of claim 16, wherein the daily amount administered is divided into at least 2 separate doses per day.

27. The method of claim 17, wherein the daily amount administered is divided into at least 2 separate doses per day.

28. The method of claim 18, wherein the daily amount administered is divided into at least 2 separate doses per day.

29. The method of claim 19, wherein the daily amount administered is divided into at least 2 separate doses per day.

30. The method of claim 20, wherein the daily amount administered is divided into at least 2 separate doses per day.

31. The composition of claim 4 wherein the flavonoid percentage is substantially greater than about 6 to 8 percent.

32. The composition of claim 5 wherein the flavonoid percentage is substantially greater than about 6 to 8 percent.
